(19) United States
(12) Patent Application Publication
(10) Pub. No.: US 2009/0062671 A1
(43) Pub. Date: Mar. 5, 2009

(54) PERIODIC SAMPLING OF CARDIAC SIGNALS USING AN IMPLANTABLE MONITORING DEVICE

(76) Inventors: Brian P. Brockway, Shoreview, MN (US); Andres Belalcazar, St. Paul, MN (US)

Correspondence Address: FISH & RICHARDSON P.C., P.O. BOX 1022, MINNEAPOLIS, MN, 55440-1022, US

(21) Appl. No.: 12/185,459

(22) Filed: Aug. 4, 2008

Related U.S. Application Data
(60) Provisional application No. 60/953,675, filed on Aug. 2, 2007.

(51) Int. Cl. A61B 5/046

(52) U.S. Cl. 600/518; 600/515

(57) ABSTRACT

In a method of diagnosing an atrial fibrillation or atrial flutter condition, a monitoring device implanted in a subject acquires strips of a subcutaneous ECG signal of a predetermined length. The strips are acquired at regular, periodic intervals, and the timing of when the strips are acquired is not triggered by analysis of the subcutaneous ECG signal by the monitoring device. The acquired subcutaneous ECG strips are stored in memory of the implanted monitoring device, and transmitted from the implanted monitoring device for receipt by an external analysis system. In the external analysis system, the received subcutaneous ECG strips are processed to generate information for an assessment of an atrial fibrillation or atrial flutter burden for the subject.

### CROSS REFERENCE TO RELATED APPLICATIONS

This application claims priority from U.S. Provisional Patent Application Ser. No. 60/953,675, filed Aug. 2, 2007, and titled “Periodic Sampling of Cardiac Signals Using an Implantable Monitoring Device,” which is incorporated by reference in its entirety.

### TECHNICAL FIELD

This disclosure relates to periodic sampling of cardiac signals.

### BACKGROUND

The human cardiovascular system is responsible for receiving oxygen-deprived blood into the heart from the venous system of the body, delivering the oxygen-deprived blood to the lungs to be replenished with oxygen, receiving the oxygenated blood from the lungs back into the heart, and delivering the oxygenated blood to the body via the arterial vasculature. This process is regulated within the heart by electrical pulses that control operation of the heart's receiving and pumping chambers. In a healthy heart, the sinoatrial node of the heart generates electrical pulses in a consistent and regulated fashion to regulate receiving and pumping blood in the heart's chambers. The electrical impulses propagate as activation wavefronts across the atria, the upper chambers of the heart, and cause cells of the atria to depolarize and contract, which forces blood from the atria to the ventricles, the lower chambers of the heart. The ventricles receive the blood from the atria, and the wavefront, after passing through the atrioventricular node and moving to the Purkinje system, moves to cells of the ventricles causing the ventricles to contract and pump the blood to the lungs and to the rest of the body.

In some patients, cardiac arrhythmias can disrupt the normal operation of the cardiac system. Atrial fibrillation is one type of cardiac arrhythmia, and involves an abnormal heart rhythm in the right and left atria. During atrial fibrillation, the heart's normal electrical impulses are disrupted, which can result in disorganized electrical impulses and irregular heart beats. The disorganized electrical impulses can cause erratic motions of the heart's chambers, and can adversely impact the timing and synchronization associated with normal blood movement through the heart and to the body. As a result, blood may pool in chambers of the heart, and may eventually form blood clots therein. A catastrophic event, such as a stroke, can occur if the clot dislodges and migrates to the brain and causes an interruption in oxygenated blood supply to the brain, for example.

It is known that some cardiac arrhythmias can be detected by measuring, recording, and analyzing cardiac electrical signals, such as an electrocardiogram (ECG) signal. Because rhythm abnormalities can be episodic and occur randomly, it is often helpful to evaluate the patient's rhythm by measuring the ECG signals over an extended period of time when the patient is ambulatory. This often involves attaching electrodes externally to a patient's skin, sensing the electrical signals that comprise the ECG signal, and recording the ECG signal on a recording device worn on the patient's body. In this case, the ECG sensing is controlled by the external recording device, to which the external electrodes are connected through leads. The external skin electrodes can be uncomfortable for the patient, however. Patient compliance issues with wearing an external recording system for more than about 2 weeks can lead to a low diagnostic yield and may render such systems impractical for longer-term monitoring.

As another example, implanted cardiac pacemakers with one or more leads extending into the patient's heart may measure an electrical signal, referred to as an electrogram, using sense electrodes, where at least one of the sense electrodes is positioned within the heart (endocardial). Implantation of a pacemaker with leads extending into the heart, however, is a non-trivial medical procedure with significant associated risks, and typically is not performed until a patient has exhibited symptoms indicative of an abnormally slow heart beat and a physician has diagnosed the abnormality and recommended the therapy. By the time this occurs, some patients may have more serious cardiac problems. Also, the population of patients experiencing some degree of atrial fibrillation may be different from the population of patients implanted with a pacemaker.

Implantable monitoring devices that can measure a subcutaneous ECG signal are known, and have been used to measure ECG signals for the purpose of diagnosing causes of syncope and, more recently, for detecting atrial fibrillation. These devices record a subcutaneous ECG signal in response to a patient-initiated activation using an external hand-held device that wirelessly communicates with the implantable monitoring device. A detection algorithm executing within the implantable device also attempts to automatically determine when an asymptomatic arrhythmia (e.g., atrial fibrillation) is in progress and may also trigger capture of ECG signal data during all or portions of the time surrounding the occurrence of the arrhythmia. The recorded ECG information is then analyzed, within the implantable device, to confirm or deny the presence of the abnormal rhythm.

Atrial fibrillation and atrial flutter are cardiac arrhythmias that can often be paroxysmal. That is, the atrial fibrillation or atrial flutter may be episodic, including occurring infrequently, and perhaps only for short durations of time. Further, it is common for the arrhythmias to be asymptomatic, so that the arrhythmia's occurrence may go unnoticed by the patient for lack of associated patient-discernable symptoms. For these reasons, it may be difficult to detect and capture evidence of atrial fibrillation or atrial flutter during an external skin electrode capture session, as the probability of an atrial fibrillation or atrial flutter event occurring during the session may be remote, and may go unnoticed or undetected if it does occur.

The details of one or more implementations are set forth in the accompanying drawings and the description below. Other features, objects, and advantages will be apparent from the description and drawings, and from the claims.

### SUMMARY

Disclosed herein are devices, systems, and techniques that can be used to monitor, from a subcutaneous implant location in the body of a subject, one or more physiologic cardiac signals by periodically sampling the signal to obtain “strips” of data that may be useful for diagnosing instances of one or more paroxysmal or persistent cardiac anomalies, including atrial fibrillation or atrial flutter. The sampling techniques disclosed herein may be implemented by an implantable subcutaneous diagnostic device powered at least in part by a battery, and may permit diagnosis of the cardiac anomaly in a power-efficient and accurate manner, such that the implantable device's battery life may be extended and the resulting information provided may have high sensitivity and specificity.

In a first general aspect, a method of diagnosing an atrial fibrillation or atrial flutter condition includes acquiring, using a monitoring device implanted in a subject, strips of a subcutaneous ECG signal of a predetermined length. The strips are acquired at regular, periodic intervals, and the timing of when the strips are acquired is not triggered by analysis of the subcutaneous ECG signal by the monitoring device. The method also includes storing the acquired subcutaneous ECG strips in memory of the implanted monitoring device, and transmitting the acquired subcutaneous ECG strips from the implanted monitoring device for receipt by an external analysis system. The method further includes processing, in the external analysis system, the received subcutaneous ECG strips to generate information for an assessment of an atrial fibrillation or atrial flutter burden for the subject.

In various implementations, the predetermined length of the strips may be in a range of about 3 to 120 seconds, or in a range of about 3 to 30 seconds. The regular, periodic intervals may have a programmable length in a range of about 3 to 120 minutes, or in a range of about 3 to 30 minutes, or in a range of about 3 to 10 minutes. The assessment of an atrial fibrillation or atrial flutter burden may be performed by the external analysis system, or by a human. The monitoring device may enter a low power mode of operation between acquisition of successive strips of the subcutaneous ECG signal. The processing may include estimating a duration of an atrial fibrillation or atrial flutter episode. The information may include, for a given time period, an estimation of time within the period that the subject experienced atrial fibrillation or atrial flutter.

In a second general aspect, an implantable monitoring device for implantation in a subject includes sense electrodes for sensing a subcutaneous ECG signal. The implantable monitoring device also includes circuitry that causes strips of the subcutaneous ECG signal of a predetermined length to be acquired using the sense electrodes. The circuitry causes the strips to be acquired at regular, periodic intervals, and the timing of when the strips are acquired is not triggered by analysis of the subcutaneous ECG signal by the implantable monitoring device. The implantable monitoring device further includes memory in which the strips of the subcutaneous ECG signal are stored, and a transmitter that transmits the acquired strips for receipt by an external analysis system for processing, in the external analysis system, the received strips to generate information for an assessment of an atrial fibrillation or atrial flutter burden for the subject.

In various implementations, the predetermined length of the strips is in a range of about 3 to 120 seconds, or in a range of about 3 to 30 seconds. The regular, periodic intervals have a programmable length in a range of about 3 to 120 minutes, or about 3 to 30 minutes. The implantable monitoring device may further include circuitry that causes the device to enter a low power mode of operation between acquisition of successive strips of the subcutaneous ECG signal.

In a third general aspect, a system for detecting atrial arrhythmia in an ambulatory subject includes an implantable monitoring device. The implantable monitoring device includes sense electrodes for sensing a subcutaneous ECG signal, and circuitry that causes strips of the subcutaneous ECG signal of a predetermined length to be acquired using the sense electrodes. The circuitry causes the strips to be acquired at regular, periodic intervals, and the timing of when the strips are acquired is not triggered by analysis of the subcutaneous ECG signal by the implantable monitoring device. The implantable monitoring device also includes memory in which the strips of the subcutaneous ECG signal are stored, and a transmitter that transmits the acquired strips. The system also includes a remote computing device that includes a receiver for receiving the transmitted strips, and an analysis module to generate information for an assessment of an atrial fibrillation or atrial flutter burden for the subject.

In various implementations, a human may provide the assessment of an atrial fibrillation or atrial flutter burden for the subject by reviewing the generated information.

Some implementations may include one or more of the following advantages: sensitivity of atrial fibrillation or atrial flutter detection may be improved; specificity of atrial fibrillation or atrial flutter detection may be improved; battery life of an implanted monitoring device may be extended; memory usage within an implanted monitoring device may be reduced; more powerful algorithms, operating on external equipment, for detecting atrial fibrillation or atrial flutter may be used; a sufficient quantity of data may be acquired with an implantable device to facilitate accurate atrial fibrillation or atrial flutter analysis, while still operating the implantable device in a power-efficient manner; atrial fibrillation or flutter may be diagnosed sufficiently early for timely therapeutic intervention; analysis results may be confirmed or rejected by a human, such as a physician.

The details of one or more implementations are set forth in the accompanying drawings and the description below. Other features, objects, and advantages will be apparent from the description and drawings, and from the claims.

### BRIEF DESCRIPTION OF THE DRAWINGS

FIG. 1 is a diagram of an exemplary system for obtaining and analyzing subcutaneous ECG signals, which involves an implantable device that periodically samples and records biological information.

FIG. 2 is a block diagram of an implantable device in accordance with an exemplary implementation and that can be used in the system of FIG. 1.

FIG. 3 is a block diagram of an exemplary implementation of a programmable device that may be used in the device of FIG. 2.

FIG. 4 is a timeline showing an exemplary periodic sampling implementation.

FIGS. 5-6 are flow charts of exemplary processes that can be used to periodically sample a cardiac signal.

FIGS. 7-8 are exemplary reports that can be produced using data acquired according to a periodic sampling process.

FIG. 9 is a flow chart of an exemplary process that can be used to detect atrial fibrillation or atrial flutter conditions.

FIG. 10 is a timeline showing exemplary strip representations and exemplary arrhythmia duration estimates.

Like reference symbols in the various drawings indicate like elements.

### DETAILED DESCRIPTION

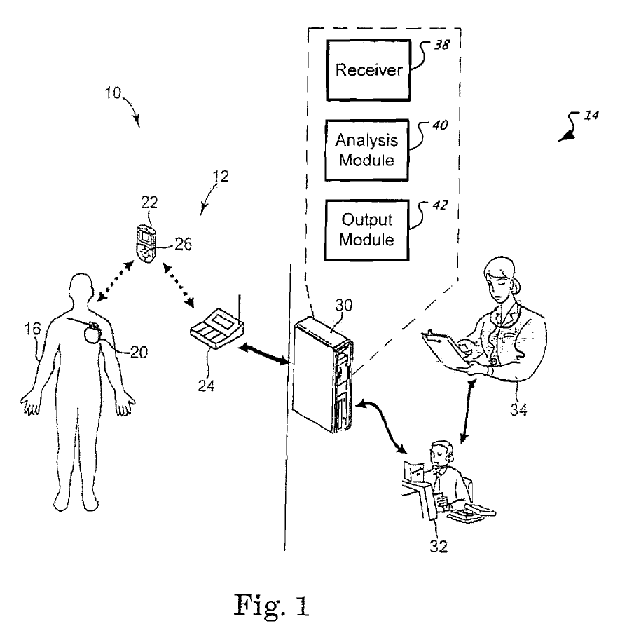

FIG. 1 is a diagram of an exemplary system 10 for obtaining and analyzing subcutaneous ECG signals, which involves an implantable device 20 that periodically samples and records biological information. According to an implementation, the device 20 may be implanted in a subject (e.g., human or animal) and may be used for regular, periodic, ongoing, and automatic recording of a subcutaneous ECG signal, or alternatively of one or more other physiologic signals. The device 20 may periodically sample and record an ECG signal, may store the recorded information within memory of the device 20, and may transmit the information to an external processing device, where it may be processed to generate information for an assessment of an atrial fibrillation or atrial flutter burden for the subject. Sampling of the subcutaneous ECG signal may be performed for a predetermined duration of time, with the sampled data constituting a “strip” of data. Timing of when the strips are acquired may not be triggered by analysis of the subcutaneous ECG signal by the monitoring device, according to some implementations. In some implementations, the timing of when the strips are acquired may be triggered, for example, by a timer or clock within the implantable device 20, and may be independent of the ECG signal, or of a present state of the ECG signal (or other physiologic signal).

Several types of arrhythmias may be diagnosed using the techniques described herein. For example, atrial fibrillation and/or atrial flutter may be diagnosed, and may be diagnosed whether the arrhythmia is paroxysmal (e.g., intermittent) or persistent (e.g., remaining until electrically or chemically cardioverted). In some implementations, atrial tachycardia, ventricular tachycardia, or other cardiac anomalies, including other types of atrial arrhythmias, may also be detected.

The device 20 may be used to record an ECG signal at regular, pre-specified intervals, which may be programmable, and may record for a pre-specified period of time, which may also be programmable, during each interval. Uploads of recorded data may occur periodically at predetermined intervals, or may occur on command, as will be described more fully below. The device 20 may similarly download information from an external device, such as operating parameters or adjustments to operating parameters, patient information, commands to perform an operation, programming updates, and the like. The implementation shown in FIG. 1 is illustrative, and many variations are possible.

The implantable device 20 may be subcutaneously implanted in a patient 16, according to an implementation. In one implementation, the device 20 may be disposed between the skin of the patient and a frontal extent of the patient's rib cage. The device 20 may include a housing, in which various electronics are housed, and a lead that extends from the housing. The lead may include an elongate lead body and one or more remote electrodes attached to the lead body and electrically connected through the lead body to circuitry within the housing. One or more sense electrodes, in some implementations, may be located on an outside surface of the housing and electrically connected to circuitry within the housing. In some implementations, the lead body and remote electrode, after implantation, remain outside of the heart or any of the heart chambers. That is, the lead body may not extend into a chest cavity of the patient 16, or into a cavity of the patient's heart, but rather may remain disposed in a subcutaneous position. In some implementations, the lead body may extend into a body vessel, such as into the subclavian vein, for example (e.g., to measure impedance or ECG from a location within the vessel). In some implementations, the device 20 does not include any leads that extend from the housing of the device 20. Rather, two or more sense electrodes on an outside surface of the housing can be used to sense a physiologic signal, such as a subcutaneous ECG signal.

The implanted device 20 may periodically sample a subcutaneous ECG waveform using two or more sense electrodes located under the patient's skin but outside of the patient's heart. The periodic sampling may be performed for a predetermined duration of time (with sampled data constituting a “strip” of physiologic signal) at regular, predetermined intervals, and the sampled signals (strips) may be stored in the device and later transmitted to an external processor for analysis, or for analysis by a human. The information may be analyzed, for example, for the presence of atrial fibrillation, atrial flutter, or any of the arrhythmias referred to above. For simplicity, the discussion below will be described with reference only to atrial fibrillation and atrial flutter diagnosis, but the techniques, systems, and devices described herein can be used to detect any of the conditions listed above.

The FIG. 1 system 10 includes patient environment 12 and service environment 14, illustrated in FIG. 1 as a first environment to the left of the vertical line in the figure and a second environment to the right, respectively. In an implementation, the patient environment 12 includes a patient 16 provided with an implantable monitoring device (IMD) 20 that is configured to collect biological data from the patient 16, a programmable external activator 22 configured to electronically communicate with (i.e., receive and transmit data from/to) the IMD 20, and a base station 24 in telemetric communication with the activator 22. In an implementation, the IMD 20 transmits stored data to the activator 22, which concurrently or at some later time may transmit the data to the base station 24. The base station 24 may be located in the patient's house, for example, and the activator 22 may be worn or carried by the patient 16 as the patient 16 goes about daily activities, according to an implementation. In some implementations, the IMD 20 may transmit recorded data directly to the base station 24.

The base station 24 may be configured to transmit data collected by IMD 20 (and possibly relayed through activator 22), over a link or network as described below, to a remote monitoring station where it may be analyzed and monitored for an indication of cardiac abnormalities, including an indication of atrial fibrillation or atrial flutter. In an implementation, the IMD 20 and activator 22 are “paired” when manufactured by programming a unique IMD identification number into a memory location of activator 22. Communications between the IMD 20 and activator 22 may include the unique identification number, for example, which may be checked by the activator 22 against the stored identifier for validation. In this manner, activator 22 is configured to recognize and communicate with a single, specific, and identifiable IMD 20.

Service environment 14 includes service system 30 configured to remotely receive the data collected by IMD 20. In various implementations, the service system 30 may include a receiver 38 to receive the data strips collected by IMD 20, an analysis module 40 that may assess one or more of the received strips for an indication of atrial fibrillation or atrial flutter, and an output module 42 that may produce a report that can be used to assess an occurrence of atrial arrhtyhmias. In some cases, the report can include a graphical display that depicts an amount of time that the patient 16 experienced atrial arrhythmias over a predetermined period, as will be described more fully below.

The service system 30 may be located at a hospital or a service center, for example. The service system 30, which may be a remote computing device, may receive the collected data in a number of ways, depending upon the implementation. In some implementations, the service system 30 receives the data from the base station 24; in alternative implementations, the service system 30 receives the data from the activator 22, or directly from the IMD 20. In some cases, the patient may visit the service environment 14, and data may be uploaded at that time. In some implementations, the service system 30 may be partitioned into two or more components or engines, where the two or more components or engines have separate physical remote locations. In each case, the components or engines may be implemented at least partially in computing devices, as will be described below. For example, a first engine may be located at one remote facility, and may receive collected strips or data (e.g., from device 24) and may automatically process the strips or data for indications of atrial arrhythmia (e.g., atrial fibrillation, atrial flutter, atrial tachycardia) in manners that will be discussed in more detail below, and a second component or engine may be located at a service center facility and may receive results from the first engine or component, where the results may include analysis of the strips or data and optionally the strips or data themselves. In this implementation, a technician may review the results or data, as will be described below. For simplicity, the discussion below will assume that the service system 30 is not partitioned into two or more components or engines, and receives the data from the base station 24.

A service technician 32 and/or a physician or other medical personnel 34 may be enabled to access the data collected by IMD 20 after the data are transmitted to service system 30. Depending upon the implementation, the base station 24 may transmit the information to the service system 30 immediately upon receipt of the information from the activator 22, or may wait until a predetermined volume of data has been received, or may transmit at periodic intervals, or may transmit on request from the service system 30. Base station 24 of patient environment 12 may be communicably linked to service system 30 of service environment 14 via any appropriate communication link or network. For example, the devices 24, 30 may be communicably linked by a land-based telephone line system, by a wireless communication network or system, by a wide-area network (WAN), local area network (LAN), the Internet, or combinations of the above. Service technician 32 and medical personnel 34 may have access to service system 30. In some implementations, service system 30 is a server or other computing device that includes one or more processors that can execute application program instructions, and can execute tasks defined by the instructions. The service system 30 may include memory for storing application programs and memory for storing received data, including data received from the base station 24. The service system 30 may include an application program that when executed analyzes recorded data from the IMD 20 to detect cardiac abnormalities, such as atrial fibrillation or atrial flutter. In some implementations, service system 30 is a desktop personal computer, a laptop computer and/or a handheld computing device such as a personal digital assistant (PDA), a mobile phone, a wearable processing device, or the like. In some implementations, the service system 30 may send a message to another electronic device, such as a mobile phone, pager, PDA, etc., that the physician or medical personnel 34, or patient 16, may carry or monitor.

The service system 30 may analyze the received physiologic data for an indication of arrhythmia, atrial fibrillation, atrial tachycardia, atrial flutter, atrial arrhythmia, focal atrial tachycardia, ventricular tachycardia, or other cardiac anomalies using various detection techniques. For example, in the case of a monitored ECG signal, the ECG signal data may be monitored for a characteristic or feature of the ECG signal data that may indicate atrial fibrillation or atrial flutter. Rhythm abnormalities, including an irregular cardiac rhythm, can indicate atrial fibrillation or atrial flutter. For example, an irregular R-R interval can be an indicator of atrial fibrillation or atrial flutter. Also, an absence of a P-wave from the ECG cycle, perhaps replaced by unorganized electrical activity, may be an indicator of atrial fibrillation or atrial flutter. The periodic sampling techniques disclosed herein may be well suited for signal acquisition that may permit timely detection of the conditions described above, while acquiring the data in a power-efficient fashion that does not unduly consume battery power, so that battery life may be extended. Further, because additional and higher performance computing hardware and software may be available in the external computing system 30, as compared to within the implantable device 16, for example, more sophisticated techniques may be used for identification of atrial fibrillation and atrial flutter detection, in some implementations. As such, sensitivity and specificity of detection may be improved as compared to approaches that attempt to assess atrial fibrillation on-board the implantable device.

In various implementations, the algorithm or algorithms for detecting atrial arrhythmia, atrial fibrillation, atrial flutter, or other cardiac anomalies may be implemented on the service system 30, on the base station 24, or on the handheld device 22. In some cases, the detection algorithm may be implemented within the implantable device 20. For example, in some implementations a detection algorithm executing within the implantable device 20 may analyze acquired strips for indications of atrial fibrillation or atrial flutter. In some examples, an algorithm executing within the implantable device 20 operates in an auto-detection mode for detecting atrial arrhythmia, and does not rely on external processing outside of the implantable device 20. That is, some implementations use an automatic, on-board detection approach. In various implementations, the approach can involve analyzing regular, periodically acquired strips, or automatically detecting arrhythmia on the physiologic signal, and possibly recording at or around the corresponding time. In some implementations, the device 20 combines automatic on-board detection of arrhythmia in the monitored signal with the regular, periodic strip acquisition techniques discussed herein. In various implementations, one of the above devices performs a portion of the detection algorithm, and another of the devices performs another portion of the algorithm. For simplicity, the discussion herein will assume that the analysis is performed at the service system 30, as by one or more application programs executing on the service system 30, which may be a remote computing device as described above.

The service system 30 may produce a report that summarizes the analysis, and may include an assessment of the patient's cardiac health. For example, the report may include one or more statistics concerning an atrial fibrillation or atrial flutter episode or episodes occurring over a particular monitoring period. Statistics of interest may include a number of atrial fibrillation or atrial flutter episodes detected, duration of each of the episodes or a composite measure of duration across episodes (e.g., mean, median, standard deviation, etc.), intensity of the episodes (assessed, for example, based on a severity of a rhythm abnormality), progression of the episodes, and the like. As another example, for a given monitoring period, a duration of time that the patient experienced atrial fibrillation or atrial flutter may be provided. In some cases, the report can include a risk assessment value that describes a likelihood that the patient will suffer an adverse cardiac event. In some cases the report can include a therapy suggestion. In some implementations, the report includes graphical information, such as one or more signal waveforms. The waveforms may correspond to ECG signals sensed by the IMD 20, for example, and may be analyzed by the physician 34 or technician 32 in some cases. In some cases, the graphical information can present data associated with the acquired strips, such as duration information relating to atrial fibrillation or atrial flutter episodes.

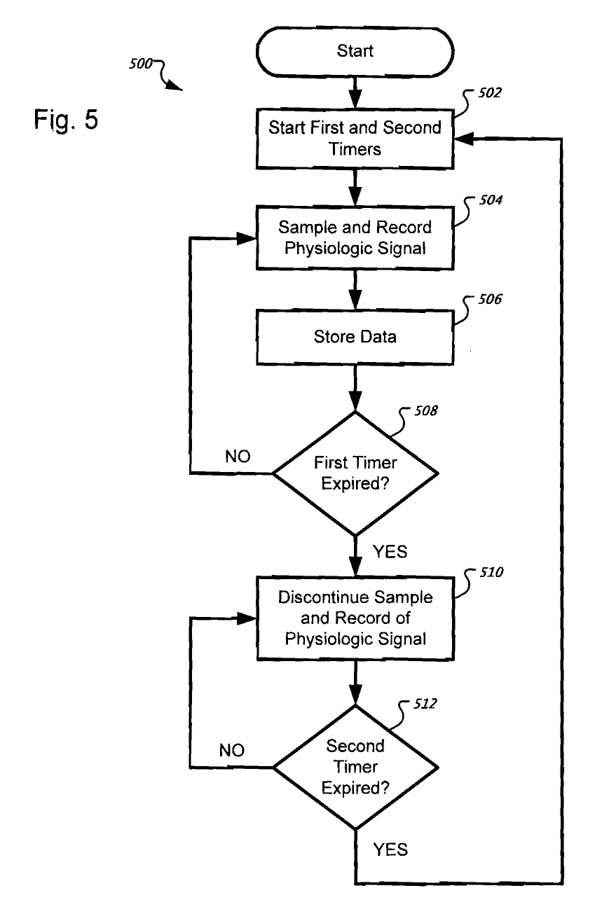

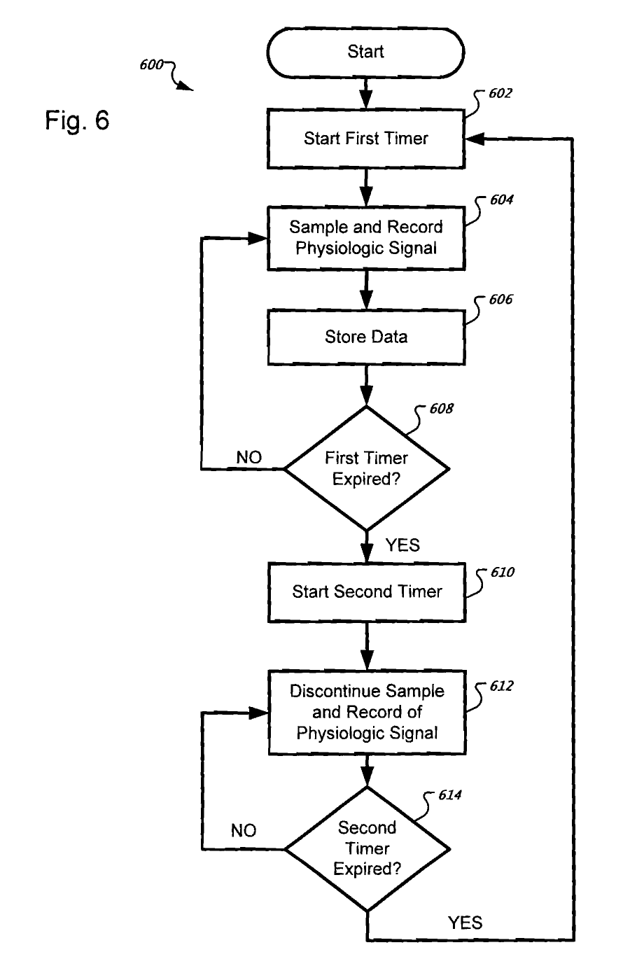

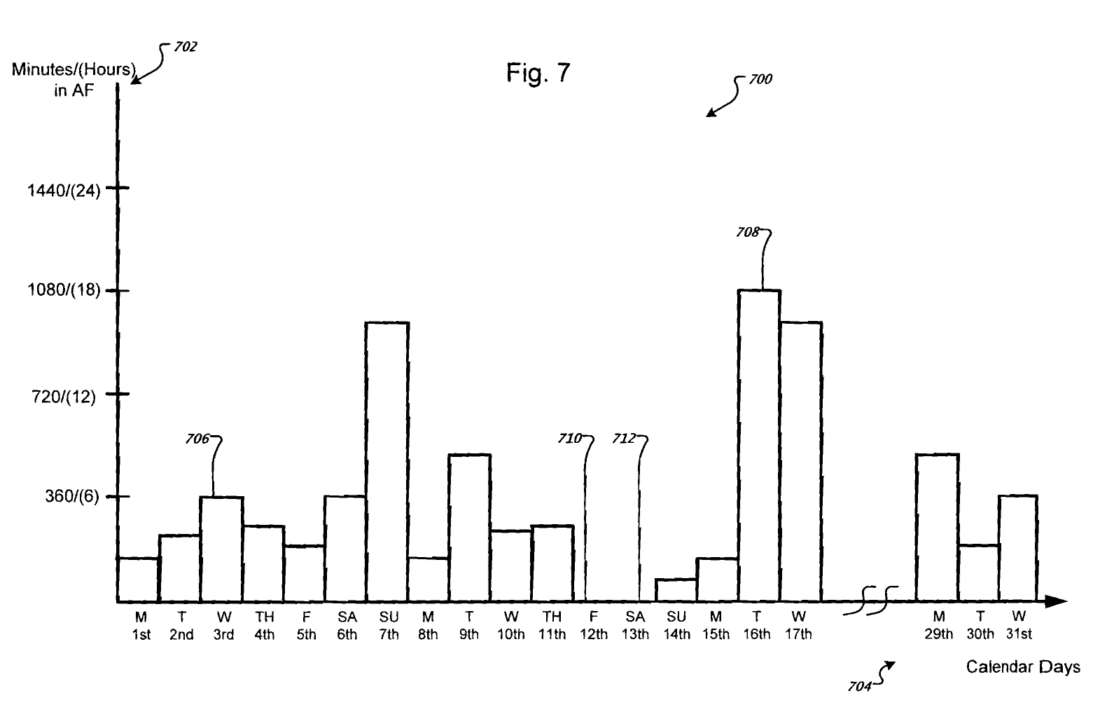

Physicians or service technicians may be interested in an amount of time that the patient experiences atrial fibrillation or atrial flutter per day, per week, per month, per year, or over any other appropriate period. FIG. 7 is an exemplary report 700 that can be produced using data acquired according to a periodic sampling process. For example, using one of the periodic sampling processes discussed above, or discussed below with reference to FIG. 5 or FIG. 6. The report 700 may be provided on a display screen of an electronic device, for example, or may be provided in printed format.

A vertical axis 702 has units of minutes in atrial fibrillation or atrial flutter, and also includes a parenthetical indication of the corresponding number of hours. A horizontal axis 704 has units of days, which are labeled according to day of the week and day of the month in this example. The exemplary report 700 is in the form of a bar graph, where a bar corresponding to each day on the horizontal axis 704 indicates, based on the height of the bar, an estimate of the number of minutes that the patient experienced atrial fibrillation or atrial flutter over the course of the day. For example, on Wednesday the 3^{rd}, the patient experienced atrial fibrillation or atrial flutter for about 360 minutes, or 6 hours, during the corresponding 24-hour period, as indicated by the bar 706 associated with that day. Similarly, on Tuesday the 16^{th}, the patient experienced atrial fibrillation or atrial flutter for about 1080 minutes, or 18 hours, as indicated by bar 708, and on Friday and Saturday the 12^{th} and 13^{th}, respectively, the patient did not experience atrial fibrillation or atrial flutter at all, as indicated by the absence of bars 710, 712 for those days. Details concerning atrial fibrillation or atrial flutter episode duration estimation will be further described below.

In this fashion, a physician or service technician may view the report 700 and quickly assess patient condition or trends in patient condition with regard to atrial fibrillation or atrial flutter. For instance, a general increasing trend over the first seven days of the month in FIG. 7 may be cause for concern, and may warrant therapy initiation or modification in some instances, such as being prescribed or increasing dosage of an anticoagulant medication, as will be discussed in further detail below. In general, if the patient is experiencing an unacceptably high number of atrial fibrillation or flutter episodes, or spending an unacceptably large number of hours over a period in atrial fibrillation or flutter, the patient may benefit from administration of an anticoagulant medication.

Also, general decreasing trends may indicate that the patient is improving and may reduce the dosage of or be taken off medications, in some cases. For example, if the patient does not experience atrial fibrillation or flutter for several consecutive days, or only experiences an acceptably low number of episodes or duration over a time period, the patient may be taken of a currently prescribed therapy in some implementations. Reports covering any appropriate time period can be produced (e.g., one day, two days, several days, a week, two weeks, one month, one quarter, one year, or longer). The reports, or statistics that can be derived from the data and/or the reports, can be used to assess a degree of recurrence of atrial fibrillation or flutter, so that decisions concerning therapy or medications may be made. The techniques may be useful in acquiring data for determination of chronic conditions or non-chronic conditions.

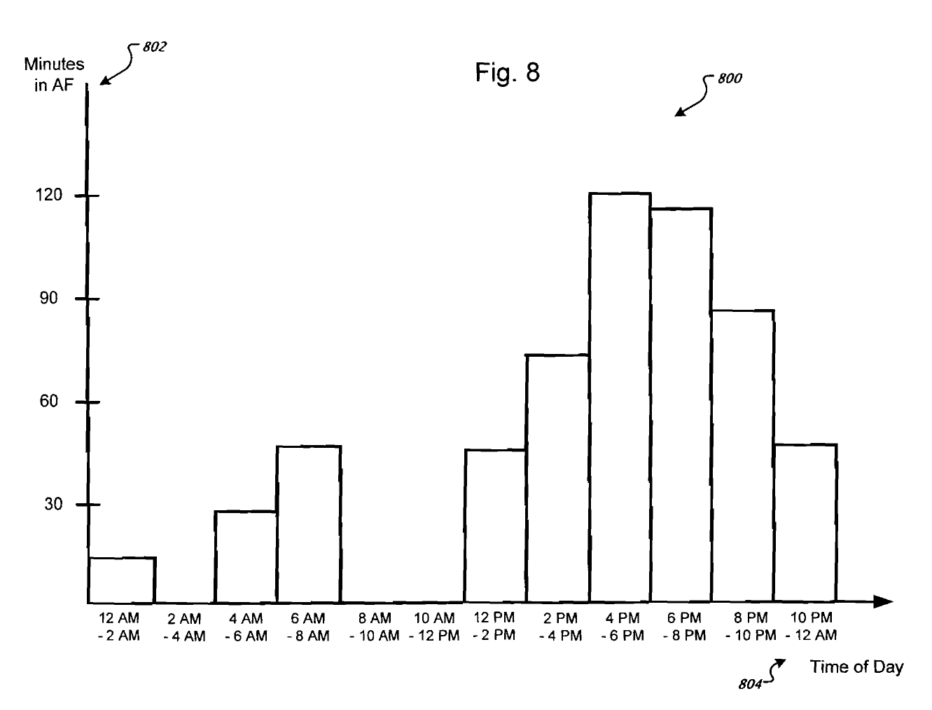

FIG. 8 is another exemplary report 800 that can be produced using data acquired according to a periodic sampling process. FIG. 8 is similar to FIG. 7 in that it depicts the time a patient experiences atrial fibrillation or flutter, but here the time period covered is one day. A vertical axis 802 has units of minutes, and a horizontal axis 804 has units of time of day, in two-hour blocks.

In some implementations, the technician 32 or physician 34 may perform one or more analysis steps to analyze the collected physiologic data. For example, in some cases, computer-implemented detection algorithms may be unable to determine with sufficient confidence a presence or absence of an arrhythmia that may lead to adverse effects as described above. In these situations, the data or information associated with the data may be sent to the technician 32 or physician 34 for further analysis.

Tiered approaches can also be used. For example, the remote computer system (e.g., system 30) may analyze all of the collected data, and the technician 32 or physician 34 may analyze a subset of the collected data or a report pertaining to all or a subset of the collected data. In some cases, redundant analysis measures can be included where the technician 32 may review all or a significant percentage of the received data, or a report generated by the system 30 pertaining to the data, and the physician reviews data as appropriate. In various implementations, the physician 34 may limit review to data (or a corresponding report) suggestive of atrial fibrillation, atrial flutter, or other cardiac anomalies, or to data that may be inconclusive and which may be better analyzed by the physician. In these cases, the technician 32 or physician 34 may confirm a presence of an arrhythmia and take an action, such as notifying the patient or physician, or authorizing or modifying a therapy, if appropriate.

In an implementation, IMD 20 is a surgically implanted device configured to periodically capture and selectively record both symptomatic (i.e., patient detected) and asymptomatic (i.e., non-patient detected, or IMD 20 detected) ECG information. In some implementations, the IMD 20 may be programmed to sample and record an ECG signal for a predetermined period of time, such as between 3 and 120 seconds (e.g., 3, 5, 10, 15, 20, 25, 30, 45, 60, 90, or 120 seconds), periodically and regularly at predetermined intervals, such as once every 3 to 120 minutes (e.g., once every 3, 4, 5, 7, 7.5, 8, 10, 12, 15, 20, 25, 30, 60, 90, or 120 minutes). This may provide flexibility, as the IMD 20 may be programmed with values that permit period sampling for durations at intervals that provide sufficient data for detecting one or more particular cardiac abnormalities, while still, because of the periodic and limited sampling, realizing conservative power consumption and corresponding reduced battery drain, along with prudent memory-management and usage.

Factors impacting the choices of sample time duration and periodic intervals, as described above, can include: 1) obtaining sufficient data for analysis such that atrial fibrillation or atrial flutter may be detected and diagnosed; 2) obtaining such data frequently enough that associated atrial fibrillation or atrial flutter detection and diagnosis may permit therapeutic interventions (described below) to be initiated prior to formation of one or more blood clots caused by pooled blood in one or more heart chambers due to the atrial fibrillation or atrial flutter; 3) desire to conserve battery power to extend battery life of the device; and 4) desire to minimize on-device memory usage to conserve power and permit devices having smaller form factors to be used, among others. These considerations can at times conflict. For example, sampling continuously or frequently for long durations can allow large amounts of data to be collected for analysis, but may cause the device 20 to consume battery power at an unacceptably high rate or require memory capacities that may be larger than desirable (e.g., for size or cost reasons). Conversely, an implementation that samples very infrequently or for only short durations, despite perhaps being efficient regarding power consumption, may result in insufficient data collection for effective diagnosis of cardiac anomalies, such as atrial fibrillation or atrial flutter.

Reliable detection of atrial fibrillation or atrial flutter by analyzing a subcutaneous ECG signal may require a certain quantity of sampled data. Given that an atrial fibrillation or atrial flutter episode is in progress, data should be obtained to cover a sufficient sample duration for analysis to determine that the episode is occurring. The sufficient sample duration may vary for different patients, and there may be many different views as to the length of an appropriate sample duration for subcutaneous ECG data acquisition. Sufficient duration of sample periods or strip lengths to acquire subcutaneous ECG data for reliable atrial fibrillation or atrial flutter detection may range from about three to five seconds on the low end to about 120 seconds on the high end. For example, at a given sample frequency, three seconds of sampled subcutaneous ECG data may in some cases be sufficient to detect an indicator of atrial fibrillation or atrial flutter, such as an irregular rhythm. In other cases, a sample duration of five seconds may be required to detect such an indicator. In yet other cases, ten, fifteen, twenty, thirty seconds, or more may be required. More reliable results may be obtained with longer sample periods, for example, but collecting additional data over longer sample periods and storing the data may consume more internal memory of the device, and may consume more power. In various implementations, IMD 20 may be programmed to sample and record an ECG signal for sample durations of between 3 and 120 seconds (e.g., 3, 5, 10, 15, 20, 25, 30, 45, 60, 90, or 120 seconds).

In some implementations, predetermined sample durations or strip lengths may be specified in terms of a number of cardiac cycles (e.g., three, four, five, eight, ten, fifteen, sixteen, twenty, thirty, thirty-two, forty, fifty, sixty, sixty-four, one-hundred, one-hundred-twenty-eight, two-hundred, two-hundred-fifty-six, etc.) rather than by a time length in seconds. Physiologic data (e.g., ECG data or biological data) acquired during a particular sample period or sample duration may be referred to as a “strip” of data. For example, in implementations where an ECG signal is monitored, the ECG signal data sampled during one five second sample period comprises an ECG strip of length five seconds, for a predetermined sample duration of five seconds.

Regarding the choice of periodic interval, continuous monitoring or monitoring very frequently using short periodic intervals may not be practically feasible, as battery life may be unacceptably short due to higher power consumption, and memory capacity requirements may become unacceptably excessive. Still, the period should be chosen such that at least all or almost all of the longer duration atrial fibrillation or atrial flutter events may be detected, as these may be more dangerous and more likely to cause blood pooling, clotting, and heighten the risk of stroke. With more frequently occurring atrial fibrillation or atrial flutter events, or those of shorter duration, choosing a periodic interval that is relatively short may provide a better chance of detecting a larger number of such events. Physicians, in addition to being interested in whether or not atrial fibrillation or atrial flutter is occurring, may also be interested in how often such events occur, the duration of the events, or a composite amount of time the patient spends in atrial fibrillation or atrial flutter. As such, it is desirable to detect as many atrial fibrillation or atrial flutter events as actually occur, even if they last for only a short duration, and if they are of a longer duration (e.g., spanning two or more periodic intervals), to determine a duration estimation for the events.

In some implementations, atrial fibrillation or atrial flutter event duration may be estimated. One method of estimating duration of an event involves counting the number of contiguous periodic intervals over which an indicator of atrial fibrillation or atrial flutter (e.g., irregular rhythm) is observed, and multiplying the count by the corresponding interval length. In some implementations, it may be assumed that atrial fibrillation or atrial flutter is present over an interval if an indication of atrial fibrillation or atrial flutter is observed in the strips that bound the interval. For example, if the interval length is fifteen minutes—so that the physiologic signal is sampled for a predetermined duration once every fifteen minutes—and if the signal indicates atrial fibrillation or atrial flutter over six consecutive intervals, a duration estimate of the corresponding event may be ninety minutes (fifteen minutes per interval multiplied by six intervals). Many variations are possible in estimating, using the acquired strips, atrial fibrillation or atrial flutter event durations, or in estimating an aggregate time that a patient experienced arrhythmias over a particular monitoring period.

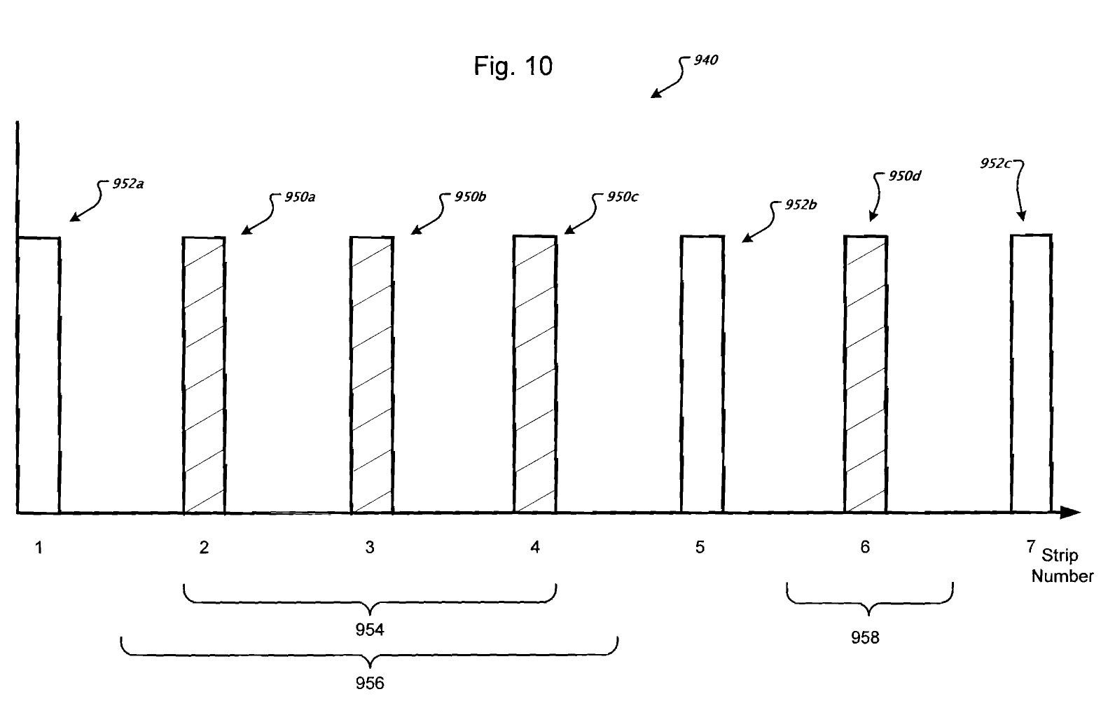

FIG. 10 is a timeline 940 showing exemplary strip representations and exemplary arrhythmia duration estimates. Two types of strip representations are shown in the timeline 940: strips 950 that indicate atrial fibrillation or atrial flutter (e.g., because they exhibit one or more features representative of an arrhythmia); and strips 952 that do not indicate atrial fibrillation or flutter. The strips are also numbered one through seven along a horizontal axis that generically labels the strips numerically. The strips 950a, 950b, 950c, 950d that indicate atrial fibrillation or flutter are shown as shaded for ease of identification, while the strips 952a, 952b, 952c that do not indicate atrial fibrillation or atrial flutter are not shaded. The strip length may correspond to any appropriate sample duration, as discussed herein, and similarly interval length may be any appropriate duration. As shown in the timeline 940, the strips are acquired at regular, periodic intervals.

As mentioned above, in some implementations it may be assumed that an arrhythmia duration includes an interval if an indication of the arrhythmia is observed in the strips that bound the interval. For example, because strips 950a and 950b indicate atrial fibrillation or flutter, some implementations may assume that the arrhythmia is present over the interval between the strips (i.e., the interval between, or between and including, strip 2 and strip 3). As shown in FIG. 10, strip 4 (950c) also indicates atrial fibrillation or flutter, so one estimate of a duration for the corresponding atrial fibrillation or flutter event includes the time spanned by the strips that indicate the arrhythmia and the intervals between the strips. This duration estimate 954 is shown as beginning with strip 2 (950a) and ending with strip 4 (950c). Alternative estimates are possible, as will now be described with another example.

As described above, in some implementations strip acquisition may occur independent of a state of the physiologic signal. That is, the physiologic signal may not be analyzed to determine when the signal should be sampled and recorded. Rather, the strips may be acquired at regular, periodic intervals, according to some implementations. With reference to FIG. 10, the arrhythmia that is first observed in strip 2 (950a, shaded), but not in strip 1 (952a, not shaded), may actually begin at some time between strip 1 and strip 2. Similarly, the arrhythmia may actually end some time between strip 4 (where it was observed) and strip 5 (where it was not observed). As the actual times that arrhythmias begin and end may be random with respect to the periodic sampling intervals, in some implementations a duration estimate for an arrhythmia may assume that the arrhythmia begins or ends at a point halfway between the strip where it was observed and the adjacent strip where in was not observed. Duration estimate 956 is an example of a duration estimate calculated in this fashion, and may represent an alternative estimate to duration estimate 954. In similar fashion, a duration estimate 958 may correspond to an atrial fibrillation or flutter event associated with strip 6 (950d).

As can be seen with duration estimates 956 and 958, the duration estimates, in intervals, correspond to the number of strips that indicate atrial fibrillation or atrial flutter. For example, the event corresponding to strip 6 (950d) was detected in a single strip, and the duration estimate 958 spans the equivalent of one interval period, from the midpoint of the interval between strips 5 and 6 to the midpoint of the interval between strips 6 and 7. Also, for the event detected in the three strips 2, 3, and 4, the duration estimate 956 runs from the midpoint between strips 1 and 2 to the midpoint between strips 4 and 5—a duration that represents three interval periods. Thus, for a given monitoring period (e.g., one day, several days, one week, two weeks, thirty days, one month, a few months, one year, etc.), an estimate of an aggregate time that the patient spent in atrial fibrillation or atrial flutter over the course of the monitoring period may be number of strips that indicate atrial fibrillation or flutter multiplied by the periodic interval length (e.g., where the periodic interval length equals the sample period, or strip length, plus an inactivity period between consecutive sample periods). In this case, an estimate of aggregate time that the patient spent in atrial fibrillation or atrial flutter is four interval lengths, because four strips (strips 2, 3, 4, and 6) indicate atrial fibrillation or atrial flutter. The estimate of four interval lengths may correspond to a sum of duration estimates 956 and 958, for example.

A recent study showed a substantial increase in risk of stroke for patients that spent 5.5 hours or more, over a twenty-four hour period, in atrial fibrillation. More specifically, it was found that when patients experienced atrial fibrillation for less than 5.5 hours in each twenty-four hour period over the course of a month, there was no noted increase in likelihood of stroke as compared to patients that did not experience atrial fibrillation. However, patients that experienced, over a thirty day period, at least one twenty-four hour period with 5.5 hours or more of atrial fibrillation, were 2.2 times as likely to suffer a stroke. This study highlights why detecting and diagnosing atrial fibrillation episodes may be useful for physicians. For example, if indications of atrial fibrillation are detected in a sufficient number of strips over a particular time period, the physician may take an appropriate action. As one example, a therapy regimen may be timely prescribed, if appropriate.

Referring again to FIG. 7, because the patient experienced more than 5.5 hours of atrial fibrillation within a 24-hour period, the patient may be at increased risk of stroke. Indeed, in this example, the patient experienced more than 5.5 hours of atrial fibrillation on several days (e.g., 3^{rd}, 6^{th}, 7^{th}, 9^{th}, 16^{th}, 17^{th}, 29, and 31^{st}). Similarly, with respect to FIG. 8, the patient experienced well over 5.5 hours of atrial fibrillation for the 24-hour period depicted.

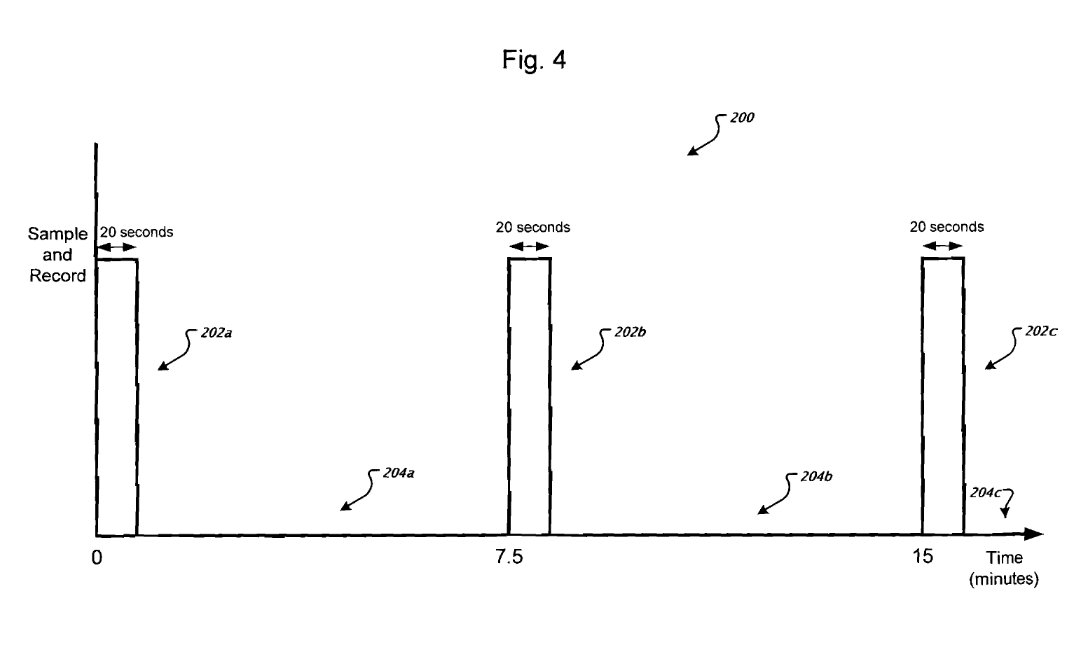

As described above, in various implementations, IMD 20 may be programmed to sample periodically at predetermined intervals, such as once every three to one-hundred-twenty minutes (e.g., once every 3, 4, 5, 7, 7.5, 8, 10, 12, 15, 20, 25, 30, 45, 60, 90, or 120 minutes) for a predetermined period of time. The intervals may be programmed or defined in various ways. For example, in some cases the interval may be programmed or defined as a time between consecutive sample periods (described below in more detail with reference to FIG. 6). In other cases, the interval may be programmed or defined to include the sample period, such that each interval includes a sample period and an inactive or non-sample period, where the sample period and the inactive period together comprise the interval (described below in more detail with reference to FIGS. 4-5).

There are different views on how long it takes a blood clot to begin forming from the onset of an atrial fibrillation event. One estimate is that blood that is pooled within a heart chamber as a result of atrial fibrillation may begin clotting within about ten minutes in some patients. Thus, under this assumption, a periodic interval of less than or equal to ten minutes may be desirable to enable possible detection of the atrial fibrillation episode prior to the onset of blood clot formation.

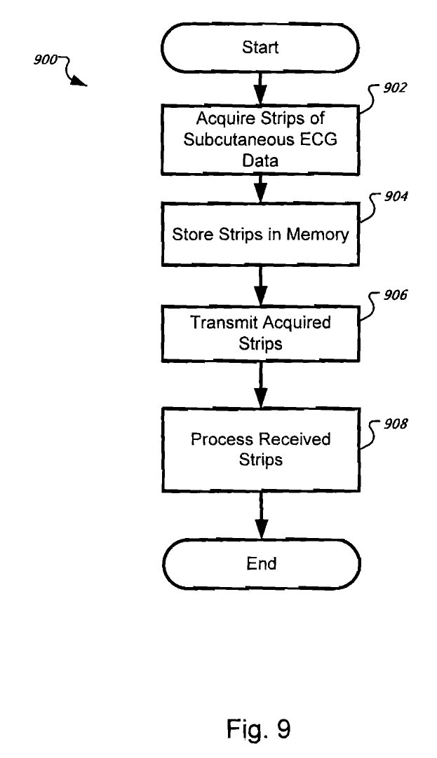

FIG. 9 is a flow chart of an exemplary process 900 that can be used to detect atrial fibrillation or atrial flutter conditions. At step 902, strips of subcutaneous ECG data are acquired. A monitoring device implanted in a subject may acquire the strips by sampling a subcutaneous ECG signal. The strips of the subcutaneous ECG signal may have a predetermined length, and may be acquired at regular, periodic intervals. In an implementation, the timing of when the strips are acquired is not triggered by analysis of the subcutaneous ECG signal by the monitoring device. Instead, timing of the strip acquisition may be triggered, for example, by a timer that manages the regular, periodic sampling.

At step 904, the strips may be stored in memory of the implantable monitoring device. At step 906, the strips may be transmitted for receipt by an external analysis system. The strips may be transmitted wirelessly, for example, using a transmitter that transmits via an antenna at RF frequency levels. At step 908, the external analysis system may process the received strips to generate information for an assessment of an atrial fibrillation or atrial flutter burden for the subject.

In various implementations, the external analysis system may be a remote computing device, or a combination of external (from the subject) devices. For instance, the implantable monitoring device may wirelessly transmit the strips, and they may be received, for example, by a handheld computing device or a home base station (such as devices 22 or 26 in FIG. 1, for example), which may then forward the strips to a remote analysis device (e.g., system 30 of FIG. 1). In some cases, the external analysis system may push some or all of the strips for review by a human operator, such as service technician 32 (see FIG. 1).

In some implementations, the external analysis system processes the strips and identifies those that show an indication of a disorganized rhythm, which may be an indicator of atrial fibrillation or atrial flutter. The system may calculate an atrial fibrillation or atrial flutter burden, using the strips or information derived from the strips. In some cases, durations of time that the subject endured atrial fibrillation or atrial flutter can be used to calculate the burden. In some implementations, a display of information may be provided, as discussed above with reference to FIGS. 7-8. This information can be used for an assessment of an atrial fibrillation or atrial flutter burden, in various implementations.

Because diagnosing cardiac arrhythmias such as atrial fibrillation or atrial flutter, including arrhythmias that are paroxysmal, can be difficult for machine-implemented algorithms in some cases, optionally a human may review the strips or information derived from the strips. Detection of the conditions described herein may be difficult, for example, because the conditions can involve rhythms that are disorganized. Detection of these conditions can be especially difficult when computing power is limited or restricted for power, size, or cost reasons, such as can sometimes be the case with implantable devices. As for human involvement, for example, a service technician or physician may review portions or all of the data. In some cases, the analysis system may evaluate a nature of a potential or suspected arrhythmia, and may ask the technician or physician to confirm. In some cases, the technician may evaluate a nature of a potential or suspected arrhythmia, and may ask the physician to confirm. In one example, the technician may review all or most of the data. The physician may provide instructions on types of data or features in the data or strips that she is interested in reviewing. In various implementations, either the analysis system or the technician may flag occurrences of the features in the data for physician review.

FIG. 4 is a timeline showing an exemplary periodic sampling implementation 200. In an implementation, the IMD 20 may sample an ECG waveform according to the sample schedule shown in FIG. 4. In this implementation, the predetermined period of time (sample length or strip length) is 20 seconds and the periodic interval length is seven-and-a-half minutes. That is, the IMD 20 samples and records the ECG signal at a predetermined frequency (e.g., 1 KHz, 500 Hz, 250 Hz, 200 Hz or 100 Hz) for twenty seconds and then waits (discontinues sampling) for seven minutes and ten seconds (i.e., seven-and-a-half minutes minus twenty seconds), then samples and records again for twenty seconds and waits for seven minutes and ten seconds, and continues in this manner, the cycle repeating every 7.5 minutes. In this fashion, the IMD 20 may record and store eight data capture episodes per hour.

FIG. 4 shows periods 202 of sample and record time, each twenty seconds in duration, during which the IMD 20 may sample the ECG signal of the patient at a rate above the Nyquist sampling rate in order to capture an ECG time series. A first sample period 202a begins at time “0,” and ends at time “20 seconds.” A second sample period 202b begins at time “7 minutes, 30 seconds,” and ends at time “7 minutes, 50 seconds.” Similarly, a third sample period 202c begins at time “15 minutes,” and ends at time “15 minutes, 20 seconds.” Only three sample periods 202 are shown in FIG. 4, but in an implementation the device 20 may periodically sample and record the ECG signal using this implementation 24 hours per day, 365 days per year. Non-sampling periods or intervals 204, during which no scheduled periodic sampling occurs, separate the sample periods 202, according to an implementation. Battery power may be conserved during each of the non-sampling periods 204a, 204b, 204c, etc. For example, electronics within the IMD 20 may be operated in a low power mode during the non-sampling periods 204, according to an implementation, or may be clocked at a lower frequency. Similarly, because circuitry within the device 20 may not sample, process, or record an ECG signal during periods 204 of inactivity, battery power consumption may be reduced during these periods 204. Sampling according to the implementation 200 shown in FIG. 4 may be referred to as burst sampling, as the ECG signal may be repeatedly sampled over a short period of time—20 seconds in FIG. 4—and then not sampled again until some longer period of time has passed (7 minutes, 10 seconds in FIG. 4, for a periodic interval of 7 minutes, 30 seconds).

The IMD 20 may sample the ECG signal using two or more sense electrodes, according to an implementation. For example, the IMD 20 may sense the ECG signal by sensing and recording a potential difference between a first electrode attached to the lead body of the device and a second electrode attached to the housing of the device. Circuitry within the IMD 20 may process the sensed signal, as will be explained more fully below. The sensed ECG signal may be converted to a digital electronic representation (for example, by an analog-to-digital converter) and stored in memory of the IMD, according to an implementation.

The recorded ECG information may be transmitted to an external monitoring station (such as service system 30, for example) for analysis, according to an implementation, perhaps using one or more intermediate devices (e.g., activator 22 and/or base station 24). The data may be analyzed for an indication of atrial fibrillation or atrial flutter, such as by detecting an irregularity in the data. Advantageously, because the IMD 20 uses periodic sampling as described above, IMD battery life may be extended and memory consumption within the IMD 20 may be minimized, which may permit IMD 20 to be smaller and less invasive, while still collecting sufficient quantities of data to enable accurate atrial fibrillation or atrial flutter detection.

The predetermined recordation period and predetermined periodic interval may be programmable, and may be set by the physician to values that the physician is comfortable with. For example, the physician may cause values for one or both of sample period duration or periodic interval to be transmitted to the device 20 wirelessly from the service system 30, base station 24, or activator 22. Alternatively, the physician may call the patient in for an office visit and may reprogram the device 20 using a wand and a dedicated device programmer, for example. In an implementation, an appropriate period and interval may be programmed at the time of implantation of the IMD 20, as by a physician. The physician 34 may later update the values by causing one or more new values to be downloaded to the device 20, as described above.

In some cases, the implanted monitoring device 20 may remain implanted within the patient 16 for long-term monitoring. The device may remain implanted for several months, one year, two years, three years, or longer. The device 20 may monitor an ECG signal by sampling subcutaneously with electrodes outside of the heart so that indicators of atrial fibrillation or atrial flutter may be detected, if they exist, by an external computing system or a physician or other medical personnel. Results from the analysis may be used, for example, to determine whether a patient is no longer experiencing atrial fibrillation or atrial flutter events, and may therefore be allowed to discontinue use of anti-coagulation medicines that the patient may have been taking. As another example, if the events are continuing to be observed or increasing in frequency, duration, or intensity, existing therapies may be modified or appropriate additional therapies may be initiated.

As described above, in an implementation, IMD 20 is configured to capture and record trending ECG waveform data based on periodic timed triggering of IMD 20. In this regard, ECG events and other biological signals can be monitored and recorded within IMD 20, which may be configured with transceiver capabilities for uploading data to activator 22. In an implementation, activator 22 uploads the biological data from the patient 16 and is configured to wirelessly transmit the data to the base station 24 when the patient 16 is, for example, within wireless fidelity (WiFi) range of base station 24. In an implementation, activator 22 is rechargeable and sized to be worn externally or carried by patient 16. In an implementation, activator 22 is a computational device including memory and programmable software that combine to enable the activator 22 to program the IMD 20, display waveforms of data collected by IMD 20 on a real-time basis, respond to patient commands, store symptomatic data collected by IMD 20, store asymptomatic data collected by IMD 20, upload data from IMD 20, download data to IMD 20, and transmit data to service system 30 via base station 24.

An implementation of activator 22 includes a patient interface 26 that is configured to enable the patient 16 to send an activation signal to selectively activate IMD 20 to record a symptomatic ECG event (e.g., an anomalous cardiac event detected by the patient 16) and, in one form of this implementation, upload information from IMD 20 to activator 22, and then to service system 30 via base station 24 during the event. In an implementation, activator 22 passively uploads ECG events recorded by IMD 20 at regular time intervals (e.g., every 10 minutes, every half hour, hourly, every 6 hours, every 8 hours, every 12 hours, daily) and transmits this data to service system 30 via base station 24. In an implementation, activator 22 is configured to receive information, such as, for example, clock synchronization information transmitted from service system 30 through base station 24, for activator 22 and/or for downloading to IMD 20.

Base station 24 may be coupled to service system 30 in a variety of suitable ways. For example, base station 24 and service system 30 may be coupled by telephone lines, wireless communication, or the Internet. Other suitable communication links between base station 24 and service system 30 may be used. Regardless of the communication link between base station 24 and service system 30, technician 32 may have access to the patient data measured by IMD 20 in some implementations. In some cases, technician 32 may provide updates to the physician 34. In some implementations, software running on the service system 30 or on a computing system used by the technician 32 may analyze the data for cardiac anomalies, and may alert the technician 32 or physician 34 when an anomaly occurs, as by an alarm or warning message, whether visible (a text or email message, or an indicator light, for example), audible, tactile (e.g., pager or mobile phone agitation), or the like.

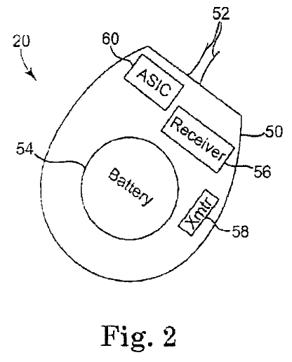

FIG. 2 is a block diagram of an implantable device 20 in accordance with an exemplary implementation and that can be used in the system of FIG. 1. In an implementation, IMD 20 includes a case 50, one or more leads 52, a battery 54, a receiver 56, a transmitter 58, and an application specific integrated circuit (ASIC) or other programmable device or component 60 contained within case 50. In an implementation, case 50 is a sealed titanium case sized to house various components of IMD 20, such as battery 54, receiver 56, transmitter 58, and ASIC 60. When implanted, IMD 20 can measure biological signals, such as ECG potentials, across leads 52 and store segments of the biological signal waveforms within ASIC 60, or within memory external to the ASIC 60 within the device 20. In an implementation, one of leads 52 is coupled to an extending lead having a remote tip electrode, and the other of leads 52 is coupled to case 50, such that an ECG potential is measurable between the remote tip electrode and case 50. In an implementation, ASIC 60 is coupled to an IMD memory device, such as a static random access memory (SRAM), which can be configured to store segments of the signal waveforms for subsequent transmission to activator 22.

Receiver 56 is configured to receive commands signals, for example, from activator 22. In an implementation, activator 22 sends an activation signal that indicates that a segment of an ECG waveform should be recorded and transmitted. When such an activation signal is received, receiver 56 can be configured to pass the activation signal to ASIC 60 so that segments of the ECG waveform are recorded. In some implementations, the waveform signals can be stored in ASIC 60 and/or an IMD memory device external from ASIC 60. The recorded segments of the ECG waveform can then be sent to transmitter 58 for transmission to activator 22. In some implementations, the signals can be transmitted directly to activator 22 rather than first storing them in ASIC 60 and/or an IMD memory device.

Once measured and transmitted, the data are available to service system 30 via the link between base station 24 and system 30. Thereafter, technician 32 or medical personnel 34 have access to the measured signals. As such, the information in the measured signals may be used by a physician to remotely diagnose a condition of patient 16, to observe and record the measured signals, and/or to further instruct IMD 20 based on the measured signals.

Because the data may be processed remotely from the IMD 20, computing devices with more powerful processing capabilities and large storage capacities can be used to analyze the received data to detect cardiac anomalies. This may permit the IMD 20 to use less power because algorithms to analyze the sampled data need not be stored and executed on the IMD 20, in some implementations. Similarly, because the IMD 20 may use periodic sampling to regularly sample and record segments of an ECG signal, as opposed to continuously sampling or engaging algorithms to detect cardiac anomalies and initiate sampling in response, IMD 20 may be operated to use less power and therefore better conserve available battery resources, which may result in extended battery life.

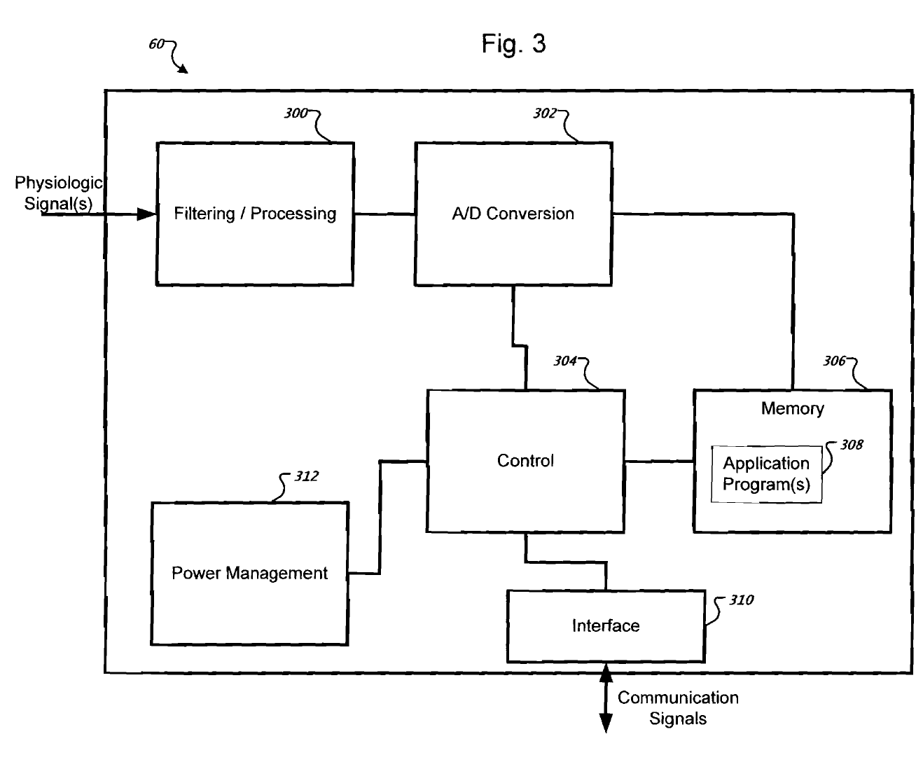

FIG. 3 is a block diagram of an exemplary implementation of a programmable device 60 that may be used in the device 20 of FIG. 2. A filtering and processing module 300 may receive one or more sensed physiologic signals, such as cardiac signals that carry information relating to a cardiac state of the patient. In an implementation, the sensed physiologic signal is an ECG signal comprising voltage potentials sensed across two or more sense electrodes. When more than two sense electrodes are employed, device 20 may have the capability of capturing two or more ECG signals, both (or all) of which can be stored and communicated out of device 20 to an external system for analysis. By capturing multiple ECG signals, the sensitivity and specificity of detection of atrial arrhythmias may be improved. In other implementations, the signal may be a blood pressure signal, a blood flow signal, or an impedance signal. The filtering and processing module 300 may filter and process the signal as is known in the art, and may provide the signal to an analog-to-digital (A/D) conversion module 302. In various implementations, such filtering and processing can include amplification or scaling of the sensed signal, as appropriate.

The A/D conversion module 302 may convert the received analog signal to a digital signal representation of the analog signal. A control module 304 can manage operations within the programmable device 60, including timing of various events and actions. In some implementations, the control module 304 includes a microprocessor or microcontroller that can execute instructions to perform tasks specified by the instructions. The instructions may be stored in a memory module 306, as within various application programs 308 that can be executed to implement the methods disclosed herein. In various implementations, the memory module 306 may comprise non-volatile memory (e.g., EPROM, flash memory, EEPROM, or various other non-volatile storage mediums familiar to those skilled in the art), volatile memory (e.g., SRAM, DRAM, SDRAM, or various other volatile mediums familiar to those skilled in the art), or a combination of non-volatile memory and volatile memory. The control module 304 may control a sampling rate of the physiologic signal, and may detect features of the sampled signal in some implementations. Also, the control module 304 may manage periods of physiologic signal sampling, and periods of inactivity (absence of sampling), including maintaining timing functions and providing control signals to facilitate the periodic sampling techniques discussed herein.

Following conversion of the analog signal to a digital signal, and in some cases post-conversion processing, the signal may be stored in memory module 306. In various implementations, a memory module external to programmable device 60 may also be used to store physiologic signal information or measured data, instructions for execution by control module 304, operational parameters of implantable device 20, patient parameters, and the like.

An interface module 310 may receive and send communications signals. For example, the interface module 310 may be coupled to receiver 56 and transmitter 58 (see FIG. 2). As described above, the implantable device 20 may transmit measured physiologic signal data (e.g., strips of data), or information pertaining to such data, wirelessly from the device for receipt by a wireless receiver external to the implantable device, and may similarly receive transmissions wirelessly from the external device.

A power management module 312 may monitor the battery 54 (see FIG. 2) periodically, or on demand, in order to determine the battery's approximate remaining life cycle. This information can be communicated to an external device in various implementations. In this manner, the approximate remaining life of battery 54 can be determined external to IMD 20 and remotely therefrom. The power management module 312 may also provide various reference voltages and/or currents for circuitry of the implantable device 20. In some implementations, the power management module 312 may manage power states for the device 20, such as controlling when one or more components of the device are in a normal power mode of operation and when they are in a low power mode of operation. Using the periodic sampling techniques disclosed herein, battery life may be extended, in some implementations.

Components or modules of the programmable device 60 described above may be combined or separated in various manners, and in some implementations one or more of the components or modules may be omitted. Similarly, in some implementations, some of the functionality described above may be implemented using discrete components, or may be incorporated into another programmable hardware device or implemented in software or firmware that may execute on a processor, whether dedicated or embedded within a programmable device. The implementation of programmable device 60 described above is exemplary, and many variations are possible.

FIG. 5 is a flow chart of an exemplary process 500 that can be used to periodically sample a cardiac signal. In various implementations, the process 500 may be executed by the implantable device 20. At step 502, first and second timers are started. The first timer may be associated with a sampling period, and the second timer may be associated with a periodic interval. In this example, the periodic interval associated with the second timer refers to an interval that includes the sample period and an inactivity period where sampling is not performed. In some cases, a single timer may be used and monitored for a first value associated with the sampling period and a second value associated with the periodic interval. In various implementations the first timer (or the first value) may be set for a time range of about 3 to 30 seconds, 3 to 60 seconds, or up to 120 seconds, and the second timer (or the second value) may be set for a time range of about 3 to 30 minutes, 3 to 60 minutes, or up to 120 minutes.

At step 504, a physiologic signal is sampled and recorded. The physiologic signal may be an ECG signal sampled or measured with subcutaneously placed electrodes. At step 506, data is stored in memory of the implantable device. In various implementations, raw signal data or processed signal data may be stored. In some implementations, the signal data may be analyzed and analysis data may be stored. If the first timer has not expired at step 508 (i.e., if the 3-120 seconds has not elapsed), the process continues at step 504 as described above. If, however, the first timer has expired at step 508, sampling and recording of the physiologic signal is discontinued at step 510. This may correspond to a period of inactivity or non-sampling for the device. In various implementations, the device may be placed into a low power mode during the period of inactivity such that a reduced amount of battery current may be required to sustain the device during the period of inactivity, and battery life may be extended. As one example, various components of the device may be clocked at a lower frequency during the period of inactivity.

If the second timer has not expired at step 512 (i.e., if the 3-120 minutes has not elapsed), the process continues at step 510 as described above. If, however, the second timer has expired at step 512, the process continues at step 502 as described above.

The implanted device 20 may transmit the stored data to an external device for analysis. In some cases, one or more intermediary external devices (e.g. device 22 or device 24) may relay the data from the implantable device 20 as discussed above. This transmission may occur at periodic intervals (e.g., once per day, twice per day, every few hours, every hour, every half hour, every 10 minutes, or according to any appropriate schedule), or may be initiated in response to receipt of a request from the external device or intermediary device.

FIG. 6 is a flow chart of an alternative exemplary process 600 that can be used to periodically sample a cardiac signal. The process 600 is similar to the process 500 of FIG. 5, except that here the periodic interval associated with the second timer refers to the inactivity period between sample periods (such as period 204 in FIG. 4), and does not include the sample period. In various implementations, the first timer (or the first value) may be set for a time range of about 3 to 120 seconds, and the second timer (or the second value) may be set for a time range of about 3 to 120 minutes.

A first timer is started at step 602. A physiologic signal is sampled and recorded at step 604, and data is stored at step 606. If the first timer has not expired at step 608 (i.e., if the 3-120 seconds has not elapsed), the process continues at step 604 as described above. If, however, the first timer has expired at step 608, a second timer is started at step 610, and sampling and recording of the physiologic signal is discontinued at step 612. If the second timer has not expired at step 614 (i.e., if the 3-120 minutes has not elapsed), the process continues at step 612 as described above. If, however, the second timer has expired at step 614, the process continues at step 602 as described above.

In an implementation, IMD 20 is implemented in a sub-8 cubic centimeters (cc) device. In this implementation, the IMD 20, which is battery-powered, may have a battery life greater than two years for monitoring physiological signals from the patient 16 or an animal. Also, this implementation of the IMD 20 has a thickness of less than 7 millimeters (mm). In addition, an implementation of IMD 20 has a telemetry distance of greater than two meters in uploading data to activator 22 and greater than two meters on downloading data from activator 22 to IMD 20. In one example implementation, IMD 20 has a battery life of five years under normal operation.

IMD 20 for monitoring patients or animals can provide highly accurate information with little or no patient compliance compared to non-invasive devices. To minimize patient complications with an implantable device, such as hematoma and infection, and to obtain sufficient patient and physician acceptance of the device, it is desirable that the implantable device be thin and have a low volume. The implementation of IMD 20 having a volume of less than 8 cc and a thickness of less than 7 mm may increase acceptability of such a device with patients and physicians.

Other devices have been previously developed that are smaller than 8 cc and have a thickness less than 7 mm, but these other devices are passive and need either a wand or a coil to be employed to provide energy to power the device at or immediately around the time that the readings are obtained. Battery-powered devices, such as IMD 20, can provide a significant boost in performance. For example, the battery can provide power for automatic signal processing, analysis, and storage of the signals being monitored. In addition, in certain implementations of IMD 20, the battery can power a receiver, a transmitter, and other communication apparatus to communicate the patient physiological information (such as ECG, EEG, pressure, temperature, activity, and the like) automatically to activator 22. This information can be transmitted from activator 22 to service system 30 via base station 24, as described above, so that a service technician 32 and physician or other medical personnel 34 are enabled to access the data collected by IMD 20 after the data is transmitted to service system 30. It may be beneficial to transmit information when the patient is sleeping or at other times when patient compliance that is needed with passive devices is difficult or impossible to obtain.

IMD 20 may permit ECG signals to be monitored while the patient 16 is ambulatory and while the patient 16 is going about normal daily activities. In this way, the system 10 may eliminate any need for patient compliance during data collection, since all data collection can be done in a manner that is transparent to the patient 16. Because system 10 collects data from an ambulatory patient, implementing system 10 allows the collection of biological signals from the patient 16 on a more frequent basis than would be practical with a method that required the patient 16 to visit a healthcare facility. Because of the paroxysmal nature of certain types of atrial fibrillation or atrial flutter, a method, such as, for example, the methods described herein, that is capable of monitoring and detecting an indicator of atrial fibrillation or atrial flutter on a more frequent basis may be likely to yield more valuable information and produce better results, including possibly earlier detection of the cardiac anomaly. As such, it may be possible to initiate therapy to address the atrial fibrillation or atrial flutter before a blood clot forms and increases the risk of a catastrophic event, such as a stroke.

Because the IMD 20 may periodically sample ECG data at regular intervals, the IMD 20 may collect a large volume of data in comparison to a system that relies on the patient to trigger a data capture, for example in response to feeling heart palpitations, or in comparison to a system that relies on algorithmic determination that an atrial fibrillation or atrial flutter event is occurring before initiating a data capture. As such, better results may be achieved with some implementations because more data may be available and the ECG data may be analyzed remotely by powerful computing machines that can execute complex analysis and detection software to identify an indicator of atrial fibrillation or atrial flutter in the captured ECG data. Moreover, ECG information that includes an indication of a paroxysmal or persistent cardiac arrhythmia condition may be captured for analysis using the present system that may be missed by other systems that rely on an algorithm to detect atrial fibrillation or a patient activation action to trigger a data capture. This may occur, for example, because such algorithms may not detect certain instances of atrial fibrillation, for example, or because a patient may similarly be unaware of such an instance or fail to initiate a data capture.

An example implementation of IMD 20 has a sub-8 cc volume, a thickness of less than 7 mm, a battery life greater than two years, and telemetry distance of greater than two meters for uploading or downloading between IMD 20 and activator 22.

If atrial fibrillation or atrial flutter are diagnosed, in some cases a physician may prescribe or modify a therapy plan for the patient. For example, because atrial fibrillation or atrial flutter may cause blood to pool and clot in the atria, increasing the patient's risk of stroke, an anticoagulant drug designed to prevent clotting may be prescribed. Warfarin and Heparin are examples of two anticoagulant medications that can be used to thin the blood, making it less prone to clotting and thereby reducing the patient's risk of suffering a stroke. The physician may also prescribe medications to control the patient's atrial arrhythmias and use ongoing information collected in the manner, for example, described herein regarding the duration and occurrence of atrial fibrillation and flutter to titrate and/or select medications and also to monitor patient compliance with the prescribed medication regimen.

Some implementations of the devices, systems, and methods disclosed herein may be useful in diagnosing a patient's risk of blood clotting caused by pooled blood in a heart chamber, as can happen when the patient experiences episodes of atrial fibrillation or atrial flutter for sufficient durations. In particular, trends or statistics observable in (from) the collected strips of data acquired using the periodic sampling techniques disclosed above, or in reports generated using the collected strips, may provide insight into adjustments to anticoagulant therapy regimens that may both provide sufficiently reduced risk of stroke and may minimize likelihood of adverse side effects that can be associated with the anticoagulants. For example, dosages of anticoagulant medications may be reduced if the results indicate that the patient is improving. On the other hand, if the patient's condition is worsening, the collected data may indicate an escalating trend, and therapy can be adjusted accordingly. Although specific implementations have been illustrated and described herein, it will be appreciated by those of ordinary skill in the art that a variety of alternative and/or equivalent implementations may be substituted for the specific implementations shown and described without departing from the scope of the present disclosure. This application is intended to cover any adaptations or variations of the specific implementations discussed herein.

For example, signals other than ECG signals can be measured, stored, and transmitted for detection of cardiac anomalies as described above. A blood pressure signal, blood flow signal, or a signal comprised of impedance measurements, any or all of which may be measured with appropriate sensors as is known in the art, can be sensed, recorded and analyzed in this fashion. Blood pressure may be measured within an artery or vein, for example, and blood flow may measured from within an artery or vein or using sensors outside of the vessel to measure flow therethrough (e.g., Doppler sensors). For example, the implantable device 20 may include one or more sense electrodes on an exterior surface of the device, or a sense port (e.g., a pressure sense port) on an exterior surface of the device. Also, the device 20 may include one or more leads (e.g., a subcutaneous lead or an intracardiac lead) or pressure sense catheters that may include various electrodes or sensors for measuring physiologic signals.

In some implementations, two or more physiologic signals may be used to assess atrial fibrillation, atrial flutter, atrial tachycardia, ventricular tachycardia, or other cardiac anomalies. For example, measured ECG signal data and measured blood pressure signal data can be used to provide a more global assessment in some cases. In some implementations, an electrogram signal can be sensed endocardially via one or more leads that extend into a patient's heart. With any of these signals, analysis for atrial fibrillation, atrial flutter, atrial tachycardia, ventricular tachycardia, or other cardiac anomalies may be conducted as described above. It is to be therefore understood that the foregoing description is intended to illustrate and not to limit the scope of the devices, methods, and systems disclosed herein. Other embodiments are within the scope of the following claims.

What is claimed is:

1. A method of diagnosing an atrial fibrillation or atrial flutter condition, comprising:
acquiring, using a monitoring device implanted in a subject, strips of a subcutaneous ECG signal of a predetermined length, wherein the strips are acquired at regular, periodic intervals, and wherein the timing of when the strips are acquired is not triggered by analysis of the subcutaneous ECG signal by the monitoring device;
storing the acquired subcutaneous ECG strips in memory of the implanted monitoring device;
transmitting the acquired subcutaneous ECG strips from the implanted monitoring device for receipt by an external analysis system; and
processing, in the external analysis system, the received subcutaneous ECG strips to generate information for an assessment of an atrial fibrillation or atrial flutter burden for the subject.

2. The method of claim 1, wherein the predetermined length of the strips is in a range of about 3 to 120 seconds.

3. The method of claim 2, wherein the predetermined length of the strips is in a range of about 3 to 30 seconds.

4. The method of claim 1, wherein the regular, periodic intervals have a programmable length in a range of about 3 to 120 minutes.

5. The method of claim 4, wherein the regular, periodic intervals have a programmable length in a range of about 3 to 30 minutes.

6. The method of claim 5, wherein the regular, periodic intervals have a programmable length in a range of about 3 to 10 minutes.

7. The method of claim 1, wherein the assessment of an atrial fibrillation or atrial flutter burden is performed by the external analysis system.

8. The method of claim 1, wherein the assessment of an atrial fibrillation or atrial flutter burden is performed by a human.

9. The method of claim 1, wherein the monitoring device enters a low power mode of operation between acquisition of successive strips of the subcutaneous ECG signal.

10. The method of claim 1, wherein the processing comprises estimating a duration of an atrial fibrillation or atrial flutter episode.

11. The method of claim 10, wherein the information includes, for a given time period, an estimation of time within the period that the subject experienced atrial fibrillation or atrial flutter.

12. An implantable monitoring device for implantation in a subject, comprising:
sense electrodes for sensing a subcutaneous ECG signal;
circuitry that causes strips of the subcutaneous ECG signal of a predetermined length to be acquired using the sense electrodes, wherein the circuitry causes the strips to be acquired at regular, periodic intervals, and wherein the timing of when the strips are acquired is not triggered by analysis of the subcutaneous ECG signal by the implantable monitoring device;
memory in which the strips of the subcutaneous ECG signal are stored; and
a transmitter that transmits the acquired strips for receipt by an external analysis system and processing, in the external analysis system, the received strips to generate information for an assessment of an atrial fibrillation or atrial flutter burden for the subject.

13. The implantable monitoring device of claim 12, wherein the predetermined length of the strips is in a range of about 3 to 120 seconds.

14. The implantable monitoring device of claim 13, wherein the predetermined length of the strips is in a range of about 3 to 30 seconds.

15. The implantable monitoring device of claim 14, wherein the regular, periodic intervals have a programmable length in a range of about 3 to 120 minutes.

16. The implantable monitoring device of claim 15, wherein the regular, periodic intervals have a programmable length in a range of about 3 to 30 minutes.

17. The implantable monitoring device of claim 12, further comprising circuitry that causes the device to enter a low power mode of operation between acquisition of successive strips of the subcutaneous ECG signal.

18. A system for detecting atrial arrhythmia in an ambulatory subject, comprising:
an implantable monitoring device that includes:
sense electrodes for sensing a subcutaneous ECG signal;
circuitry that causes strips of the subcutaneous ECG signal of a predetermined length to be acquired using the sense electrodes, wherein the circuitry causes the strips to be acquired at regular, periodic intervals, and wherein the timing of when the strips are acquired is not triggered by analysis of the subcutaneous ECG signal by the implantable monitoring device;
memory in which the strips of the subcutaneous ECG signal are stored; and
a transmitter that transmits the acquired strips; and
a remote computing device that includes a receiver for receiving the transmitted strips, and an analysis module to generate information for an assessment of an atrial fibrillation or atrial flutter burden for the subject.

19. The system of claim 18, wherein a human provides the assessment of an atrial fibrillation or atrial flutter burden for the subject by reviewing the generated information.
